(19) United States
(12) Patent Application Publication
(10) Pub. No.: US 2009/0099427 A1
(43) Pub. Date: Apr. 16, 2009

(54) MICRONEEDLE ARRAY WITH DIVERSE NEEDLE CONFIGURATIONS

(75) Inventors: Arvind N. Jina, San Jose, CA (US); Shashi P. Desai, San Jose, CA (US); Beelee Chua, Davis, CA (US); Ashok Parmar, Fremont, CA (US)

Correspondence Address: SHAY GLENN LLP, 2755 CAMPUS DRIVE, SUITE 210, SAN MATEO, CA, 94403, US

(73) Assignee: ArKal Medical, Inc., Fremont, CA (US)

(21) Appl. No.: 11/871,806

(22) Filed: Oct. 12, 2007

(51) Int. Cl. A61B 5/00

(52) U.S. Cl. 600/309

(57) ABSTRACT

The invention relates to a surface penetration device, a method to use the device, and an analyte monitor. Embodiments of the surface penetration device include a substrate with first and second surfaces, and first and second tissue piercing elements, the elements differing in configuration, but each associated with the first surface of the substrate. At least some of the tissue piercing elements have a distal and a proximal opening and a lumen extending between the openings. The proximal openings are in fluid communication with an opening in the second surface of the substrate. Embodiments of the analyte monitor include the features of the penetration device plus an analyte sensor that detects an analyte in a fluid. Embodiments of the method of penetrating tissue include providing a surface penetration device and urging the surface penetration device against a tissue surface until some of the first and second tissue piercing elements penetrate the tissue surface.

### INCORPORATION BY REFERENCE

All publications and patent applications mentioned in this specification are herein incorporated by reference to the same extent as if each individual publication or patent application was specifically and individually indicated to be incorporated by reference

### BACKGROUND OF THE INVENTION

The invention relates to tissue piercing elements that can be used in systems for and methods of monitoring analytes, such as monitoring glucose in people having diabetes.

In the discussion that follows, reference is made to certain structures and/or methods. However, the following references should not be construed as an admission that these structures and/or methods constitute prior art. Applicants expressly reserve the right to demonstrate that such structures and/or methods do not qualify as prior art.

Diabetes is a chronic, life-threatening disease for which there is no known cure. It is a syndrome characterized by hyperglycemia and relative insulin deficiency. Diabetes affects more than 120 million people world wide, and is projected to affect more than 220 million people by the year 2020. It is estimated that one out of every three children today will develop diabetes sometime during their lifetime. Diabetes is usually irreversible, and can lead to a variety of severe health complications, including coronary artery disease, peripheral vascular disease, blindness and stroke. The Center for Disease Control (CDC) has reported that there is a strong association between being overweight, obesity, diabetes, high blood pressure, high cholesterol, asthma and arthritis. Individuals with a body mass index of 40 or higher are more than 7 times more likely to be diagnosed with diabetes.

There are two main types of diabetes, Type I diabetes (insulin-dependent diabetes mellitus) and Type II diabetes (non-insulin-dependent diabetes mellitus). Varying degrees of insulin secretory failure may be present in both forms of diabetes. In some instances, diabetes is also characterized by insulin resistance. Insulin is the key hormone used in the storage and release of energy from food.

As food is digested, carbohydrates are converted to glucose and glucose is absorbed into the blood stream primarily in the intestines. Excess glucose in the blood, e.g. following a meal, stimulates insulin secretion, which promotes entry of glucose into the cells, which controls the rate of metabolism of most carbohydrates.

Insulin secretion functions to control the level of blood glucose both during fasting and after a meal, to keep the glucose levels at an optimum level. In a normal person blood glucose levels are between 80 and 90 mg/dL of blood during fasting and between 120 to 140 mg/dL during the first hour or so following a meal. For a person with diabetes, the insulin response does not function properly (either due to inadequate levels of insulin production or insulin resistance), resulting in blood glucose levels below 80 mg/dL during fasting and well above 140 mg/dL after a meal.

Currently, persons suffering from diabetes have limited options for treatment, including taking insulin orally or by injection. In some instances, controlling weight and diet can impact the amount of insulin required, particularly for non-insulin dependent diabetics. Monitoring blood glucose levels is an important process that is used to help diabetics maintain blood glucose levels as near as normal as possible throughout the day.

The blood glucose self-monitoring market is the largest self-test market for medical diagnostic products in the world, with a size of approximately over $3 billion in the United States and $7.0 billion worldwide. It is estimated that the worldwide blood glucose self-monitoring market will amount to $9.0 billion by 2008. Failure to manage the disease properly has dire consequences for diabetics. The direct and indirect costs of diabetes exceed $130 billion annually in the United States—about 20% of all healthcare costs.

There are two main types of blood glucose monitoring systems used by patients: single point or non-continuous and continuous. Non-continuous systems consist of meters and tests strips and require blood samples to be drawn from fingertips or alternate sites, such as forearms and legs (e.g. OneTouch® Ultra by LifeScan, Inc., Milpitas, Calif., a Johnson & Johnson company). These systems rely on lancing and manipulation of the fingers or alternate blood draw sites, which can be extremely painful and inconvenient, particularly for children.

Continuous monitoring sensors are generally implanted subcutaneously and measure glucose levels in the interstitial fluid at various periods throughout the day, providing data that shows trends in glucose measurements over a short period of time. These sensors are painful during insertion and usually require the assistance of a health care professional. Further, these sensors are intended for use during only a short duration (e.g., monitoring for a matter of days to determine a blood sugar pattern). Subcutaneously implanted sensors also frequently lead to infection and immune response complications. Another major drawback of currently available continuous monitoring devices is that they require frequent, often daily, calibration using blood glucose results that must be obtained from painful finger-sticks using traditional meters and test strips. This calibration, and re-calibration, is required to maintain sensor accuracy and sensitivity, but it can be cumbersome as well as painful.

At this time, there are four products approved by the FDA for continuous glucose monitoring, none of which are presently approved as substitutes for current glucose self-monitoring devices. Medtronic (www.medtronic.com) has two continuous glucose monitoring products approved for sale: Guardian® RT Real-Time Glucose Monitoring System and CGMS® System. Each product includes an implantable sensor that measures and stores glucose values for a period of up to three days. One product is a physician product. The sensor is required to be implanted by a physician, and the results of the data aggregated by the system can only be accessed by the physician, who must extract the sensor and download the results to a personal computer for viewing using customized software. The other product is a consumer product, which permits the user to download results to a personal computer using customized software.

A third product approved for continuous glucose monitoring is the Glucowatch® developed by Cygnus Inc., which is worn on the wrist like a watch and can take glucose readings every ten to twenty minutes for up to twelve hours at a time. It requires a warm up time of 2 to 3 hours and replacement of the sensor pads every 12 hours. Temperature and perspiration are also known to affect its accuracy. The fourth approved product is a subcutaneously implantable glucose sensor developed by Dexcom, San Diego, Calif. (www.dexcom.com). All of the approved devices face challenges in obtaining bodily fluid samples for monitoring glucose levels therein. What is needed and not provided by the prior art are devices, systems and methods for obtaining and monitoring bodily fluid samples in a safe, simple, reliable, cost-effective and pain-free manner.

### SUMMARY OF THE INVENTION

Various aspects of the present invention include a surface penetration device, a method by which to use a surface penetration device, and an analyte monitor.

Embodiments of the surface penetration device include a substrate having a first surface and a second surface, a plurality of first tissue piercing elements extending from and supported by the first surface of the substrate, and a plurality of second tissue piercing elements extending from and supported by the first surface of the substrate. The first tissue piercing elements have a common first configuration, and the second tissue piercing elements have a common second configuration. The second configuration is substantially different from the first configuration. At least some of the tissue piercing elements have a distal opening, a proximal opening, and a lumen extending between the distal and proximal openings. The proximal openings are in fluid communication with an opening in the second surface of the substrate.

In some embodiments of the above summarized surface penetration device, the device further includes a plurality of third tissue piercing elements extending from and supported by the first surface of the substrate, the third tissue piercing elements having a common third configuration, wherein the third configuration is substantially different with respect to both the first and second configurations.

In various embodiments of the above-summarized surface penetration device, the first tissue piercing elements may be micro-needles, they may be arranged in an array having a square format, or they may be arranged in an array having a hexagonal format.

In some embodiments of the above-summarized surface penetration device, the first tissue piercing elements include lumens and the second tissue piercing elements do not include lumens. In other embodiments, both the first and the second tissue piercing elements include lumens.

In some embodiments of the above-summarized surface penetration device, the first configuration of the first tissue piercing may variously include a slender pyramid, a broad-base pyramid, a skinny sharp needle, or a snake fang. In some embodiments, the first configuration of the first tissue piercing elements includes a slender pyramid and the second configuration of the second tissue piercing elements includes a skinny sharp needle. In some of these latter embodiments, the first tissue piercing may be surrounded by a plurality of second tissue piercing elements.

Embodiments of the analyte monitor include at least one first tissue piercing element having a first configuration, and at least one second tissue piercing element having a second configuration. The second configuration is substantially different from the first configuration. At least one of the tissue piercing elements has a distal opening, a proximal opening, and a lumen extending between the distal and proximal opening. A sensing area is in fluid communication with the proximal opening of the tissue piercing element, and an analyte sensor is adapted to detect an analyte in a fluid located in the sensing area.

Some embodiments of the above-summarized analyte monitor further include at least one third tissue piercing element extending from and supported by the first surface of the substrate. This third tissue piercing element has a third configuration, that configuration being substantially different from both the first and second configurations.

In some embodiments of the above-summarized analyte monitor, the first tissue piercing element may variously be a micro-needle, it may include a plurality of first tissue piercing elements arranged in an array having a square format, or arranged in an array having a hexagonal format. In some embodiments of the above-summarized analyte monitor, the first tissue piercing element may include a lumen and the second tissue piercing element does not comprise a lumen, and in some embodiments both the first and the second tissue piercing elements may include lumens.

Embodiments of the method of penetrating tissue include providing a surface penetration device and urging the surface penetration device against a tissue surface until at least some of the first tissue piercing elements and at least some of the second tissue piercing elements penetrate the tissue surface. The surface penetration device used in this method includes a substrate having a first surface and a second surface, a plurality of first tissue-piercing elements extending from and supported by the first surface of the substrate, the first tissue piercing elements having a common first configuration, and a plurality of second tissue piercing elements extending from and supported by the first surface of the substrate. The second tissue piercing elements have a common second configuration, the second configuration being substantially different from the first configuration. At least some of the tissue piercing elements have a distal opening, a proximal opening, and a lumen extending between the distal and proximal openings. The proximal openings are in fluid communication with an opening in the second surface of the substrate. In some embodiments of the method, the surface penetration device is urged against the tissue surface until at least some of the distal openings of the tissue piercing elements come in contact with interstitial fluid.

Other aspects of the invention will be apparent from the specification and drawings.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which.

FIGS. 1 and 2 are cross-sectional schematic views of a glucose monitoring device according to one embodiment of the invention with tissue piercing elements in place on a user's skin.

FIGS. 3-6 show exemplary substantially cylindrical needles of the present invention.

FIGS. 7(a)-7(c) show a method of forming deformed substrate layer of a glucose monitor.

FIG. 8 shows a close up view of a distal opening of a tissue piercing element in a deformed substrate layer.

FIG. 9 illustrates an exemplary deformed substrate layer defining a plurality of tissue piercing elements.

FIG. 10 shows a perspective view of the optionally disposable portion of the glucose monitor.

FIG. 11 shows an exploded view of a glucose monitoring device according to another embodiment of the invention.

FIGS. 12(a) and 12(b) are a schematic representative drawing of a three electrode system for use with the glucose sensor of one embodiment of this invention.

FIGS. 13(a) and 13(b) are a schematic representative drawing of a two electrode system for use with the glucose sensor of one embodiment of this invention.

FIG. 14 is a cross-sectional schematic view of a portion of a glucose monitoring device according to yet another embodiment of the invention.

FIG. 15 shows a remote receiver for use with a glucose monitoring system according to yet another embodiment of the invention.

FIG. 16 shows a glucose sensor in place on a user's skin and a remote monitor for use with the sensor.

FIG. 17 shows an exemplary tissue piercing element having a first configuration.

FIG. 18 shows another exemplary tissue piercing element having a second configuration.

FIG. 19 shows an exemplary array of mixed tissue piercing elements.

FIGS. 20-26 depict exemplary embodiments of various mixed tissue piercing element array patterns.

### DETAILED DESCRIPTION OF THE INVENTION

The present invention provides a significant advance in biosensor and glucose monitoring technology: portable, painless, virtually non-invasive, self-calibrating, integrated and non-implanted sensors which continuously indicate the user's blood glucose concentration, enabling swift corrective action to be taken by the patient. The invention may also be used in critical care situations, such an in an intensive care unit to assist health care personnel. While reference is made herein primarily to glucose, the sensor and monitor of this invention may be used to measure any other analyte as well, for example, electrolytes such as sodium or potassium ions. As will be appreciated by persons of skill in the art, the analyte sensor can be any suitable sensor including, for example, an electrochemical sensor or an optical sensor.

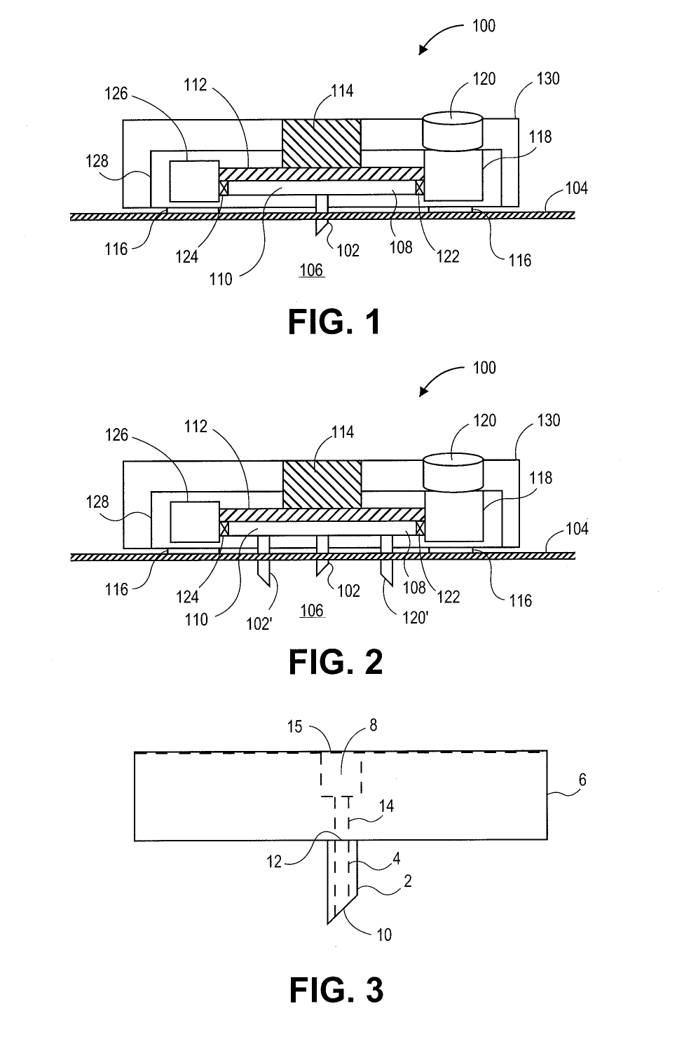

FIG. 1 shows a schematic cross-section of one embodiment of the analyte monitor. The analyte monitor 100 has one hollow needle 102 or other tissue piercing element extending through the stratum corneum 104 of a subject into the interstitial fluid 106 beneath the stratum corneum. The tissue piercing element is preferably hollow and has an open distal end, with an interior that communicates with a sensing area 110 within a sensor channel 108. Sensing area 110 is therefore in fluid communication with interstitial fluid 106 through needle 102. In this embodiment, sensing area 110 and the needle 102 are pre-filled with sensing fluid prior to the first use of the device. Thus, when the device is applied to the user's skin and the needle pierces the stratum corneum of the skin, there is substantially no net fluid transfer from the interstitial fluid into the needle. Rather, an analyte such as glucose diffuses from the interstitial fluid into the sensing fluid within the tissue piercing element as described below.

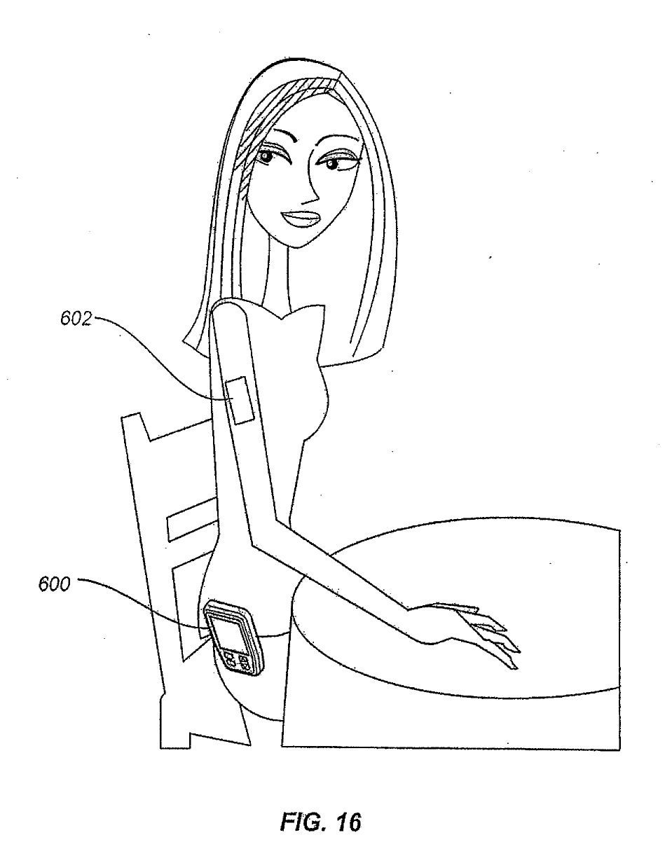

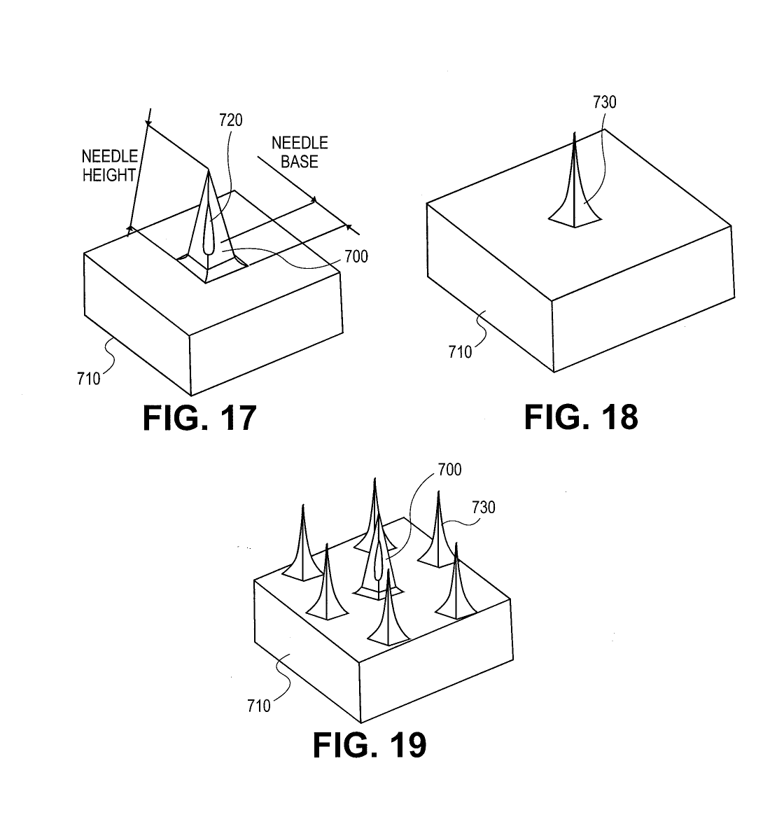

FIG. 2 shows another embodiment of the glucose monitor with a total of three (3) needles 102, 102′. The illustrated glucose monitors are not intended to be a limitation on the number of tissue piercing elements that can be used with a glucose monitor of the present invention. The glucose monitor may have one, two, three, four, or more tissue piercing elements adapted to pierce the stratum corneum. In this embodiment, the outer needles 102′ have a common configuration that is different from the configuration of the inner needle 102. In particular, outer needles 102′ are longer than and have an opposite orientation to inner needle 102. The significance of this difference is explained below in the discussion relating to FIGS. 17-26.

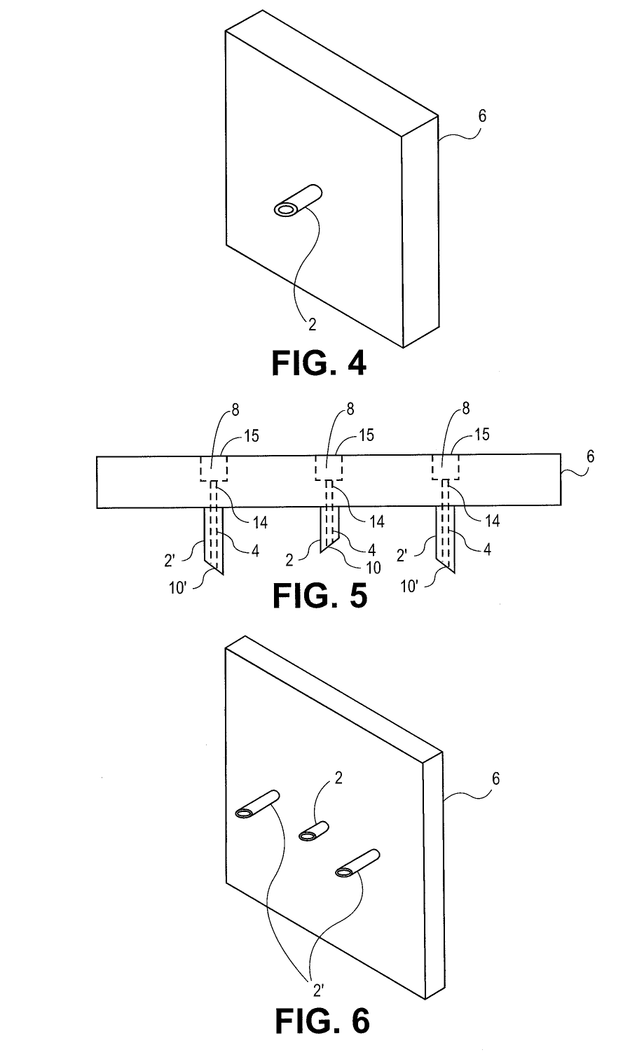

FIGS. 3 and 4 provide a side view and perspective view, respectively, of the needle shown in FIG. 1. As shown, needle 2 engages and is coupled to a substrate or chip 6 of the glucose monitor. Needle 102 is substantially cylindrical in shape and has a substantially cylindrical interior lumen 4 shown in phantom which provides a channel between the distal opening 10 and the proximal opening of the needle 12. Substrate 6 has a substrate lumen 14 shown in phantom which is in fluid communication with the interior lumen of the needle 4 and the sensing area 8.

FIGS. 5 and 6 show an alternative embodiment wherein the glucose monitor has a total of three (3) needles 2, 2′ to pierce the stratum corneum of the skin into the interstitial fluid. As used herein, “needle” or “the needle” can refer to a single needle as shown in FIGS. 3 and 4, or more than one needle, as shown in FIGS. 5 and 6. As indicated above, the outer needles 102′ of this embodiment are longer than and have an opposite orientation to inner needle 102. Again, the significance of this difference is explained below in the discussion relating to FIGS. 17-26.

FIGS. 3-6 can also show needle or needles 2, 2′ passing through the interior of, and supported by, the substrate 6. The interior lumen of the needle would comprise lumen 4, lumen 14 and area 8 in FIGS. 3 and 5. In such embodiments the proximal opening of the needle is 15.

In this embodiment a passageway can be created in substrate 6 by any method known in the art, such as, for example, etching. A needle can then be inserted into the formed passageway to position the needle in the position shown in FIGS. 3-5, such as by press fitting. The needle can be a commercially available hypodermic needle and may or may not have to be altered before placing through and into substrate 6.

The tissue piercing elements are preferably made from any metal or alloy such as a stainless steel. Other metals of which the needle can be made are iron, brass, bronze, nickel, aluminum, chrome, titanium, platinum, gold, silver, tantalum, tungsten, iridium, palladium, rhodium, ruthenium, osmium, molybdenum, or cobalt. Commercially available hypodermic needles may be used in the glucose monitor, such as those manufactured by Becton Dickinson or UltiMed Incorporated.

Exemplary tissue piercing elements and their methods of production that can be used with the present invention can be found in U.S. Pat. No. 7,076,987 to Martin et al. A commercially available hypodermic needle may need to be adapted before use with the monitors as described herein. For example, for a desired tissue piercing element length of 1 mm, it may be necessary to shorten a commercially available hypodermic needle. Other processing steps such as, for example, laser cutting, grinding, or polishing the edges may be performed as well. If the tissue piercing element is not set at a right angle in relation to the monitor however, the length of the needle could be determined based on the degree of the angle.

In this embodiment the tissue piercing element is generally substantially cylindrical in shape, as shown in FIGS. 3-6. While the tissue piercing elements in FIGS. 3-6 are shown with circular cross-sections, they are not limited to such shapes. Substantially cylindrical tissue piercing elements includes tissue piercing elements that have cross-sections that are non-circular, such as hexagonal or any other cross-sectional shape.

The distal opening of the tissue piercing element can have a tapered cut as shown in FIGS. 3-6 to allow for quick and efficient penetration of the skin. The distal tapered end can have a variety of shape designs to allow for improved penetration, such as designs described in U.S. Pat. No. 6,945,964, filed Oct. 14, 2003.

While the needles shown in FIGS. 3-6 are shown at a right angle to the substrate, the needle can be coupled to the substrate or pass through the substrate to assume any number of angles in relation to the substrate. For example, the needle can be at a 45 degree angle to the substrate such that the needle penetrates the skin at a 45 degree angle. In addition, the needles shown in FIGS. 3-6 are substantially straight. However, the needles may have a different shape such as a curved shape to allow for easier penetration in the skin. In embodiments in which multiple needles are used, the needles may have varying lengths to allow for easier penetration into the skin.

A commercial hypodermic needle is generally available in a variety of gauges ranging from, for example, 7 to 35, but a hypodermic needle with a larger or smaller gauge number can be used. Generally, a small diameter is preferred to minimize the pain a patient will feel, however, a diameter that is too small may not provide enough structural support to penetrate the stratum corneum. In some embodiments the needle can be about 28 to about 32 gauge (i.e., about 0.36 millimeters outside diameter to about 0.23 millimeters outside diameter). In other embodiments the gauge can be about 35 or smaller. Any other gauge/diameter needle may be used in the glucose monitor of the present invention.

The length of the tissue piercing element is preferably long enough to pierce the stratum corneum and come into contact with the interstitial fluid such that glucose from the interstitial fluid can diffuse through the needle as described below. Commercially available hypodermic needle can be coupled directly on the glucose monitor or through it, or can first be altered such as shortening the length to achieve a desirable length before engaging with the glucose monitor.

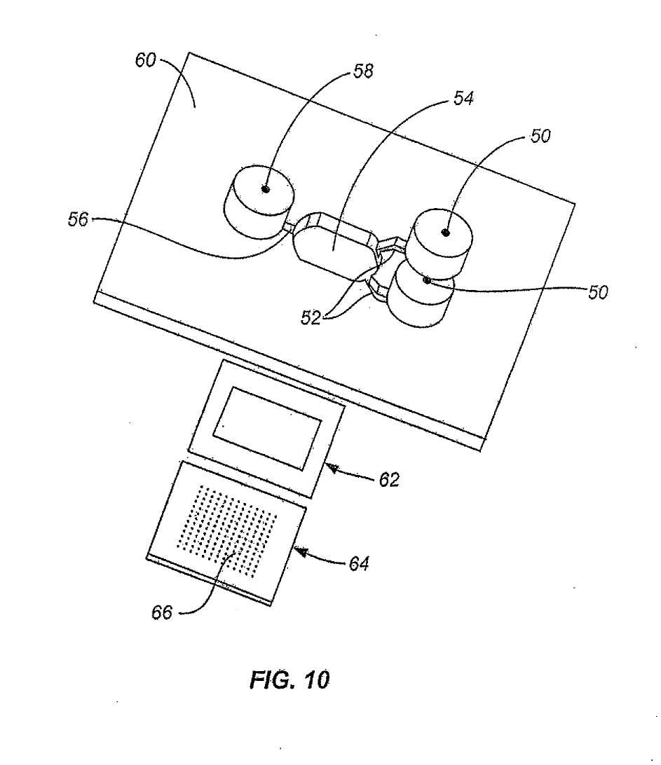

Suitable materials for the substrate include but are not limited to metals, alloys such as a stainless steel, plastic, silicon, germanium, minerals (e.g. quartz), semiconducting materials (e.g. silicon, germanium, etc.), ceramic, polymers and plastic. While the substrates as shown are in a generally rectangular shape, the substrate can be in any other shape or size as may be desirable to orient the substrate in the glucose monitor. In addition, a substrate lumen is shown in FIGS. 3-6 which can fluidly connect the interior lumen of the needle with the sensing area. The substrate lumen need not always be present and the interior lumen of the needle can be in direct fluid communication with the sensing area. The sensing area is shown in FIGS. 3-6, however the sensing area need not be located inside the substrate but can be in a separate channel above the substrate (not shown in FIGS. 3-6), shown as sensing area 208 in FIG. 10 described below.

Fabrication of a lumen in the substrate and/or the sensing area in the substrate, such as lumen 14 and sensing area 10 in FIGS. 3-6 can be achieved by, for example, without limitation, a fabrication method including dry plasma etching, wet aqueous etching, water jet drilling, solid particles ablation and photon or electron beam drilling.

The tissue piercing element can be a separate component from the substrate and can be attached to the substrate by an adhesive, glue, or other bonding technique such that the substrate lumen formed in the substrate aligns with the interior space of the needle to create a lumen extending from the distal opening of the needle to the sensing area through which the glucose can diffuse. While the substrate lumen 14 and interior lumen 4 are shown aligned in the same direction in FIGS. 3-6, the substrate lumen 14 could also form other passages for the glucose to diffuse. For example, substrate lumen 14 could form a number of right angles before connecting to the sensing area.

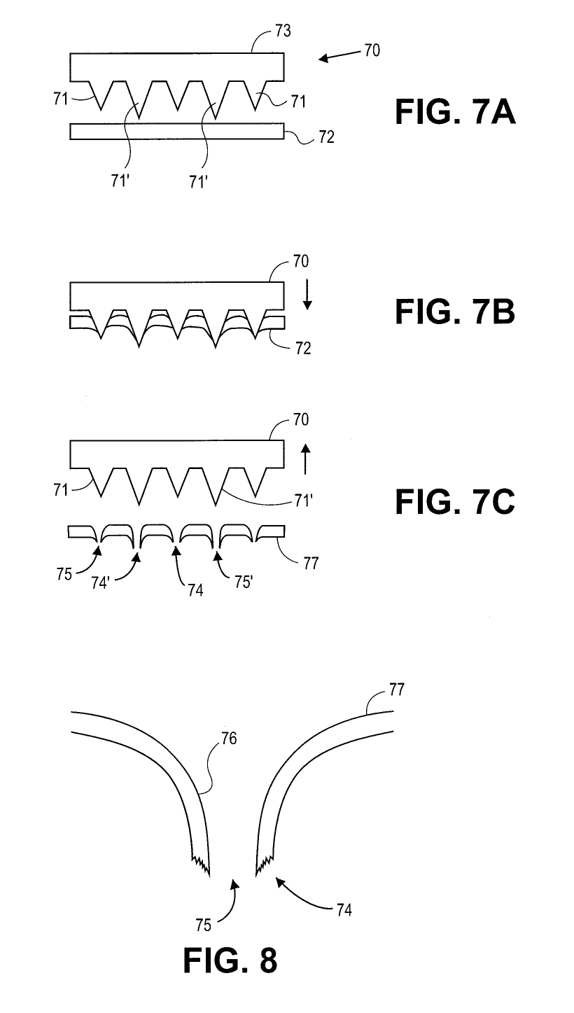

Another aspect of the invention is a glucose monitor that in some embodiments comprises a deformed substrate layer defining a plurality of tissue piercing elements. In these embodiments, each of the tissue piercing elements has a distal opening, a proximal opening and a lumen or channel extending between the distal and proximal openings. The tissue piercing elements are preferably protrusions which are integrated with and extend from one side of the substrate. An exemplary method of manufacturing the tissue piercing elements will assist in describing their structure. FIGS. 7(a)-7(c) are sectional views which show an exemplary method of producing the deformed substrate layer. Substrate actuator 70 comprises a plurality of pins or extensions 71, 71′ which extend from the base of substrate actuator 73. Substrate 72 is positioned below the substrate actuator 70. Substrate actuator 70 is lowered, in FIG. 7(b), such that pins 71, 71′ engage and puncture substrate 72 creating distal openings 75, 75′, respectively. Substrate actuator 70 is then returned to its initial position in FIG. 7(c), providing deformed substrate layer 77 defining tissue piercing elements 74, 74′.

FIG. 9 illustrates an exemplary deformed substrate layer with an array of tissue piercing elements 74, 74′ with respective distal openings 75, 75′ and deformed substrate layer 77.

By way of reference, the tissue piercing elements in this embodiment can be analogized to the rough protrusions of a cheese grater. Furthermore, the substrate actuator piercing through the substrate can be analogized to a pin puncturing a sheet of aluminum foil. FIGS. 7 and 9 illustrate two shapes the tissue piercing elements can assume based on the shape and design of the pin used to puncture the substrate. In FIGS. 7-9 the tissue piercing elements have a general volcano shape, broader at their proximal end than at the distal end. The shape of the tissue piercing element will generally depend on the size and shape of the actuator pins. In this embodiment, it can be seen in FIGS. 7(a)-7(c) that pins 71′ are longer than pins 71. Consequently, when longer pins 71′ engage and puncture substrate 72 (as shown in FIG. 7(b)), they create longer and/or wider distal openings 75′ than the openings 75 created by shorter pins 71 (as shown in FIG. 7(c) and FIG. 9). In other words, the shorter tissue piercing elements 74 have a common first configuration that is substantially different than the common second configuration of longer tissue piercing elements 74′. Again, the significance of this difference is explained below in the discussion relating to FIGS. 17-26.

In one embodiment the substrate actuator is a steel dye but can be any material capable of piercing through the substrate and create the distal openings. For example, the dye can have steel pins extending therefrom.

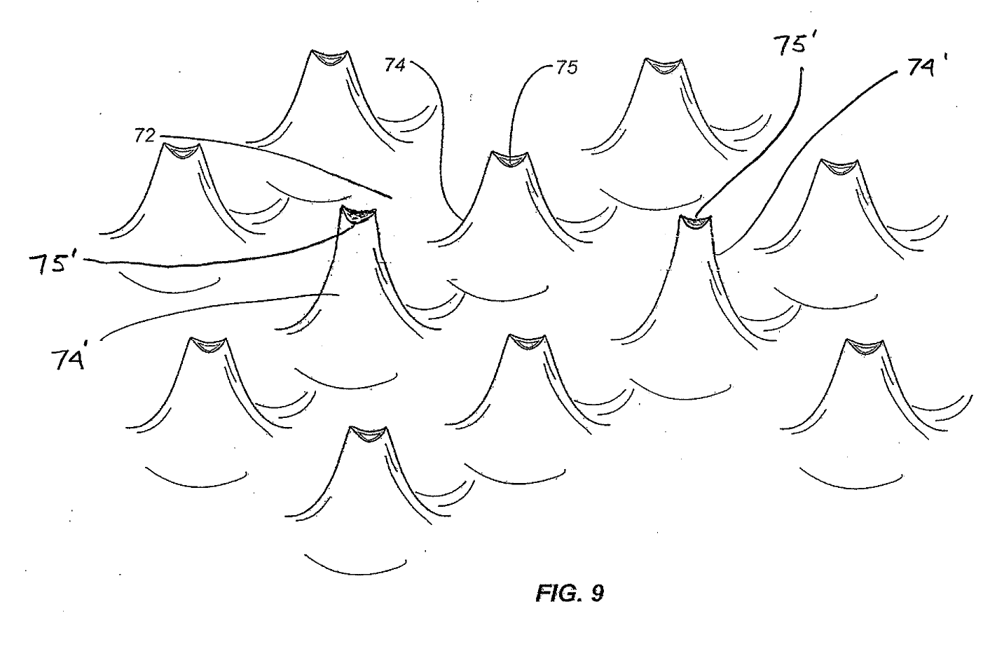

The substrate is preferably a metal sheet that can be made of any metal or alloy such as a stainless steel. Other exemplary metals that can be used alone or in combination are iron, brass, bronze, nickel, aluminum, chrome, titanium, platinum, gold, silver, tantalum, tungsten, iridium, palladium, rhodium, ruthenium, and osmium. The metal sheet is preferably of a thickness and strength such that the tissue piercing elements embedded therein are capable of piercing the stratum corneum of the skin to allow for glucose to diffuse through the distal opening of the tissue piercing elements. Similar to the tissue piercing elements described in FIGS. 3-6, the tissue piercing elements have interior lumens 76 (shown in FIG. 8) which create a fluid network between the distal openings of the tissue piercing element and the sensing area.

A deformed substrate layer can be configured to be disposed in the glucose monitor in the same or similar position as the tissue piercing elements in FIG. 3-6. The deformed substrate layer could be in the same position as the substrate such that the distal opening would be in fluid communication with sensing area.

Disposed above and in fluid communication with the sensor channel 108 shown in FIGS. 1-2 is a glucose sensor 112. In some embodiments, glucose sensor is an electrochemical glucose sensor that generates an electrical signal (current, voltage or charge) whose value depends on the concentration of glucose in the fluid within sensing area 110. Details of the operation of glucose sensor 112 are discussed below.

Sensor electronics element 114 is configured to receive an electrical signal from sensor 112. In some embodiments, sensor electronics element 114 uses the electrical signal to compute a glucose concentration and display it. In other embodiments, sensor electronics element 114 receives and transmits the electrical signal, or information derived from the electrical signal, to a remote device, such as through wireless communication. Electronics element 114 can comprise other circuitry such as an amplifier and an A/D converter which can amplify the electrical signal from the sensor and convert the amplified electrical signal to a digital signal before, for example, determining a glucose concentration or transmitting the digital signal to an external device which can then determine a glucose concentration.

Glucose monitor 100 can be held in place on the skin 104 by one or more adhesive pads 116.

Glucose monitor 100 has a novel built-in sensor calibration system. A sensing fluid reservoir 118 contains a sensing fluid having, e.g., a known glucose concentration between about 0 and about 400 mg/dl. In some embodiments, the glucose concentration in the sensing fluid is selected to be below the glucose sensing range of the sensor. The sensing fluid may also contain buffers, preservatives or other components in addition to the glucose. Upon manual or automatic actuation of a pump, plunger, or other actuator 120, fresh sensing fluid is forced from sensing fluid reservoir 118 through a check valve 122 (such as a flap valve) into sensing channel 108. Any sensing fluid within channel 108 is forced through a second check valve 124 (e.g., a flap valve) into a waste reservoir 126. Check valves or similar gating systems are used to prevent contamination.

Because the fresh sensing fluid has a known glucose concentration, sensor 112 can be calibrated at this value to set a base line. After calibration, the sensing fluid in channel 108 remains stationary, and glucose from the interstitial fluid 106 diffuses through needle 102 into the sensing area 110. Changes in the glucose concentration over time reflect differences between the calibration glucose concentration of the sensing fluid in the sensing fluid reservoir 118 and the glucose concentration of the interstitial fluid, which can be correlated with the actual blood glucose concentration of the user using proprietary algorithms. Because of possible degradation of the sensor or loss of sensor sensitivity over time, the device may be periodically recalibrated by operating actuator 120 manually or automatically to send fresh sensing fluid from sensing fluid reservoir 118 into sensing area 110.

In some embodiments there may be two or more sensing fluid reservoirs as shown in FIG. 10. A glucose monitor with two or more sensing fluid reservoirs can be calibrated at one or more different glucose concentrations, which can provide a more accurate calibration curve, which can therefore provide for a more accurate glucose concentration calculation.

FIG. 10 shows a perspective view of the optionally disposable portion of the glucose monitor. Housing 60 includes a fluidic network in which a plurality of reservoirs and channels are in fluid communication to allow for the movement of sensing fluid (or calibration fluid) from at least one sensing fluid reservoir through a sensing area and into at least one waste reservoir. Housing 60 is coupled to seal 62 which is coupled to substrate or chip 64 which comprises at least one tissue piercing element 66.

As shown, housing 60 includes sensing fluid reservoirs 50 in fluid communication with sensing fluid channels 52, which are adapted to receive sensing fluid from the sensing fluid reservoirs. Sensing fluid channels 52 are in fluid communication with sensing area or sensing channel 54. Sensing area 54 is connected to waste channel 56, which is in fluid communication with waste reservoir 58. When substrate 64 is coupled to seal 62 and seal 62 is coupled to housing 60, the at least one tissue piercing element 66 is in fluid communication with sensing area 54. While not shown, a pump and/or series of valves can be incorporated into the glucose monitor to provide for the flow of fluid from the sensing fluid reservoirs to the waste reservoir. Also not shown is an actuator which can be manually or automatically actuated and work in tandem with a pump and/or series of valves to initiate the flow of fluid from the sensing fluid reservoirs. The channels shown in FIG. 10 are intended to be optional in the glucose monitor, as the fluid can flow directly from the sensing fluid reservoirs into the sensing area, and further directly into the waste reservoirs. Similarly, one or more waste reservoirs may be incorporated into the glucose monitor.

Waste reservoirs may be or include an absorption device such as a diaper-like material to absorb waste fluids. In such embodiments the waste reservoir may not necessarily be an enclosed structure, but may simply be an absorptive material in fluid communication with the sensing area so that it can absorb waste fluids as they are moved from the sensing area.

Incorporating a plurality of sensing fluid reservoirs into the glucose monitor, as shown in FIG. 10, allows for a multiple point calibration curve to be generated during the glucose sensor calibration, which can provide a more accurate glucose concentration calculation. The sensing fluids in each of the different sensing fluid reservoirs can have different known glucose concentrations, enabling the glucose sensor to be calibrated at more than one calibration point. In general, the more calibration points that can be used to generate a relationship between the concentration of sensed glucose in the sensing area and the glucose sensor output, the more accurate the results of the glucose concentration in the interstitial fluid, and therefore the blood, may be. In some embodiments a first sensing fluid has a glucose concentration of between about 0 mg/dl and about 100 mg/dl, and a second sensing fluid has a glucose concentration of between about 100 mg/dl and about 400 mg/dl. When one or more sensing fluid reservoirs are used, the sensing fluids in each reservoir may, however, have substantially the same glucose concentration.

While in some embodiments the glucose monitor may be manually actuated to initiate the calibrating procedure, the glucose monitor can also be self-calibrating or self-actuating. For example, the glucose monitor can include a programmable component, such as a timer, that is programmed to automatically activate a pump and valve system to initiate the flow of fresh sensing fluid from any of the sensing fluid reservoirs into the sensing area. The timer can be preprogrammed, or in some embodiments the monitor includes a first housing to be worn on the skin which includes the sensor and a second housing that is separate from the first housing that can display a glucose concentration. The second housing can be adapted such that it can program the programmable component in the first housing. For example, a patient may want to program the monitor to calibrate at certain times during the day. The first housing can include a timer that can be wirelessly programmed or reprogrammed by the patient using the second housing's user interface to start the calibration at certain times.

In one embodiment of monitoring a subject's interstitial fluid glucose concentration, the method includes calibrating the glucose sensor with a plurality of different sensing fluids, which may have different concentrations of glucose. A first sensing fluid of known glucose concentration can either be moved into the sensing area upon manufacture of the glucose monitor, or can be moved from a sensing fluid reservoir into the sensing area before the glucose monitor is first used. An output from the glucose sensor is detected by the electronics element and associated with the first known glucose concentration. Any actuating technique described herein may be used to move a second sensing fluid with a second known concentration from a second sensing fluid reservoir into the sensing area, forcing the first sensing fluid into the waste reservoir. The output from the glucose sensor can then be similarly detected by the electronics element and associated with the second known glucose concentration. Using these at least two associations of glucose concentration to glucose sensor output, a calibration curve or plot can be computed to relate glucose concentration to glucose sensor output, which can then be used to determine glucose concentration of the glucose that diffuses into the sensing area from the interstitial fluid. Any number of sensing fluids, and thus calibration points, can be used to calibrate the glucose sensor. The calibrated sensor is then ready to sense a glucose concentration in the sensing area.

In embodiments where two or more sensing fluids with different glucose concentrations are used to calibrate the sensor, it may be advantageous to retain the fluid with the lower glucose concentration (such as a first concentration between about 0 mg/dl and 100 mg/dl) in the sensing area after the calibrating step, to provide for faster response times for the glucose sensing. In the method described above where the second sensing fluid has a higher glucose concentration, it may be advantageous to move a volume of fresh first sensing fluid into the sensing area after the glucose sensor output from the second sensing fluid is detected. This would move the second sensing fluid from the sensing area into waste reservoir.

In some embodiments at least one finger-stick calibration may optionally be performed or may be required to be performed at any point during the use of the monitors as described herein.

In some embodiments the glucose monitor includes a body temperature sensor. The body temperature sensor is adapted to detect the temperature of the body of the subject. As described herein, the glucose sensor senses a concentration of glucose in the sensing fluid within the sensing area. The concentration of glucose in the sensing fluid depends on the rate of diffusion of glucose molecules between the interstitial fluid in the subject and the sensing fluid in the sensing area. Diffusion is temperature dependent and as such the rate of the diffusion of glucose molecules between the interstitial fluid and the sensing fluid in the sensing area may depend on the body temperature of the subject. The rate of diffusion may increase as the body temperature increases, and may similarly decrease as the body temperature decreases. For example, a higher than normal body temperature can result in a higher rate of diffusion. Determining an accurate glucose concentration in the subject may therefore depend on knowing the body temperature of the subject, which can affect the rate at which glucose diffuses from the subject into the sensing area.

The body temperature sensor can be in the form of a patch that is worn on the skin. It can comprise an adhesive such as a hydrogel to attach to the subject's skin. It can also comprise one or more thermistors to sense the temperature of the patient's body.

The temperature sensor can be either separate from the glucose monitor or incorporated into the glucose monitor. The body temperature sensor can be in wired communication with at least one other component, such as the electronics element so that the output from the body temperature sensor can be communicated to the, for example, electronics component where it can be used in the calculation of a glucose concentration or transmitted to a housing separate from the sensor where it can be then used in the calculation of a glucose concentration. The body temperature sensor may, however, be in communication with a different component or multiple components. The body temperature sensor can, however, include a transmitter for transmitting the sensed body temperature to the glucose monitor if, for example, the body temperature sensor is a patch worn separately from the glucose monitor housing or housings.

In one embodiment the temperature sensor is incorporated into the glucose monitor and is located on the underside of the monitor, so that when the monitor is worn by the subject, the body temperature sensor is in contact with the skin. In such embodiments, a separate body temperature adhesive may or may not be used, as the body temperature sensor may contact the skin simply by pressure from the glucose monitor.

In some embodiments the glucose monitor includes a vibration assembly adapted to ease the penetration of the needle into the stratum corteum of the skin. The vibration assembly can include a vibration element such as a vibration motor which drives an unbalanced load or an off-set weight, as can be found in many commercial handheld devices such as cell phones or PDAs. The vibration element, however, can be a different type of vibratory mechanism that can initiate a vibration effect to ease the penetration of the needle into the skin, such as an ultrasonic vibrator. The vibration element can cause the vibration of one or more components of the glucose monitor.

Upon initiation of the vibration, the device can activate a separate force applicator that provides a force from the device towards the surface of the skin to assist in the needle penetration of the skin. The user, however, can simply apply pressure with, for example, the palm side of the hand from on top of the glucose monitor towards the surface of the skin when the vibratory effect occurs to assist in the penetration of the skin. In some embodiments, however, when a vibration motor is used in the vibration assembly, the vibration motor can be housed inside the glucose monitor in a configuration such that a torque results from the rotation of the motor (during the vibration) and the vibration motor causes a downward force from the glucose monitor towards the surface of the skin to assist the needle in penetrating the stratum corneum layer of the skin.

In some embodiments the monitor can include an applicator to apply the sensor pad or adhesive pad to the skin. The applicator pad may be part of the sensor device or when the monitor includes separate components, it may be included in any of the different components.

In some embodiments, the needle(s) or tissue piercing element(s) 102, reservoirs 118 and 126, channel 108, sensor 112 and adhesive pads 116 are contained within a support structure (such as a housing 128) separate from electronics element 114 and actuator 120, which are supported within their own housing 130. This arrangement permits the sensor, sensing fluid and needle(s) to be discarded after a period of use (e.g., when reservoir 118 is depleted) while enabling the electronics and actuator to be reused. A flexible covering (made, e.g., of polyester or other plastic-like material) may surround and support the disposable components. In particular, the interface between actuator 120 and reservoir 118 must permit actuator 120 to move sensing fluid out of reservoir 118, such as by deforming a wall of the reservoir. In these embodiments, housings 128 and 130 may have a mechanical connection, such as a snap or interference fit.

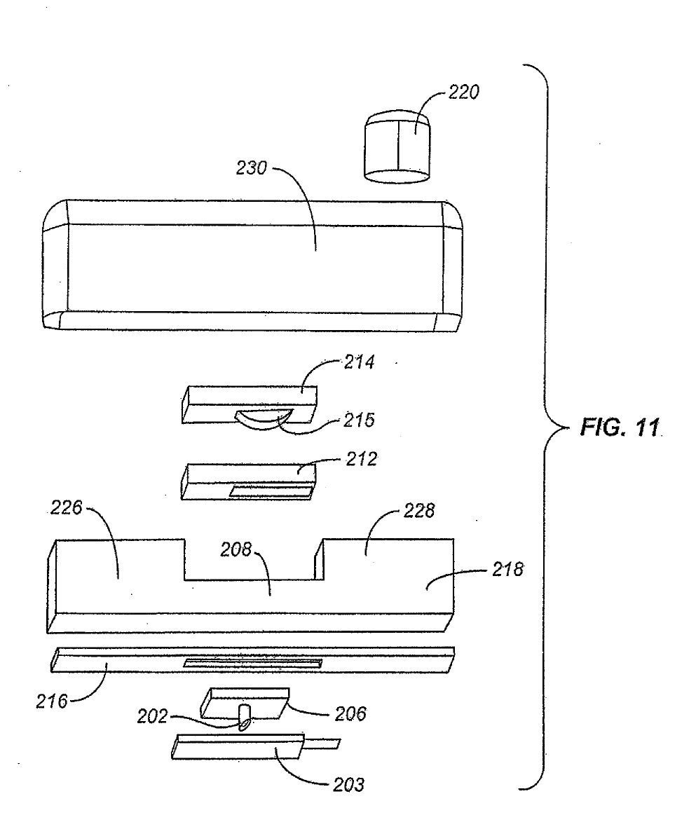

FIG. 11 shows an exploded view of another embodiment of the invention. This figure shows a removable seal 203 covering the distal end of needle 202 and attached, e.g., by adhesive. Seal 203 retains the sensing fluid within the needle and sensing area prior to use and is removed prior to placing the glucose monitor 200 on the skin using adhesive pressure seal 216. In this embodiment, needle 202, sensing fluid and waste reservoirs 218 and 226, sensing microchannel 208 and electrochemical glucose sensor 212 are contained within and/or supported by a housing 228 which forms the disposable portion of the device. A second housing 230 supports an electronics board or element 214 (containing, e.g., processing circuitry, a power source, transmission circuitry, etc.) and an actuator 220 that can be used to move sensing fluid out of reservoir 218, through microchannel 208 into waste reservoir 226. Electrical contacts 215 extend from electronics board 214 to make contact with corresponding electrodes in glucose sensor 212 when the device is assembled. While one needle is shown in FIG. 11, multiple needles having the same or different configurations may be used, for example, as shown in FIGS. 5-6. In addition, the glucose monitor of FIG. 11 may incorporate the deformed substrate layer defining a plurality of tissue piercing elements as described herein, and may replace substrate 206 and needle 202.

The following is a description of glucose sensors that may be used with the glucose monitors of this invention. In 1962 Clark and Lyons proposed the first enzyme electrode (that was implemented later by Updike and Hicks) to determine glucose concentration in a sample by combining the specificity of a biological system with the simplicity and sensitivity of an electrochemical transducer. The most common strategies for glucose detection are based on using either glucose oxidase or glucose dehydrogenase enzyme.

Electrochemical sensors for glucose, based on the specific glucose oxidizing enzyme glucose oxidase, have generated considerable interest. Several commercial devices based on this principle have been developed and are widely used currently for monitoring of glucose, e.g., self testing by patients at home, as well as testing in physician offices and hospitals. The earliest amperometric glucose biosensors were based on glucose oxidase (GOX) which generates hydrogen peroxide in the presence of oxygen and glucose according to the following reaction scheme:

Glucose+GOX-FAD (ox)→Gluconolactone+GOX-FADH₂ (red) GOX-FADH₂ (red)+O₂→GOX-FAD (ox)+H₂O₂

Electrochemical biosensors are used for glucose detection because of their high sensitivity, selectivity and low cost. In principal, amperometric detection is based on measuring either the oxidation or reduction of an electroactive compound at a working electrode (sensor). A constant potential is applied to that working electrode with respect to another electrode used as the reference electrode. The glucose oxidase enzyme is first reduced in the process but is reoxidized again to its active form by the presence of any oxygen resulting in the formation of hydrogen peroxide. Glucose sensors generally have been designed by monitoring either the hydrogen peroxide formation or the oxygen consumption. The hydrogen peroxide produced is easily detected at a potential of 0.0, 0.1, 0.2, or any other fixed potential relative to a reference electrode such as an Ag/AgCl electrode. However, sensors based on hydrogen peroxide detection are subject to electrochemical interference by the presence of other oxidizable species in clinical samples such as blood or serum. On the other hand, biosensors that monitor oxygen consumption are affected by the variation of oxygen concentration in ambient air or in any of the fluids used with the monitors as described herein. In order to overcome these drawbacks, different strategies have been developed and adopted.

Selectively permeable membranes or polymer films have been used to suppress or minimize interference from endogenous electroactive species in biological samples. Another strategy to solve these problems is to replace oxygen with electrochemical mediators to reoxidize the enzyme. Mediators are electrochemically active compounds that can reoxidize the enzyme (glucose oxidase) and then be reoxidized at the working electrode as shown below:

GOX-FADH₂ (red)+Mediator (ox)→GOX-FAD (ox)+Mediator (red)

Organic conducting salts, ferrocene and ferrocene derivatives, ferricyanide, quinones, and viologens are considered good examples of such mediators. Such electrochemical mediators act as redox couples to shuttle electrons between the enzyme and electrode surface. Because mediators can be detected at lower oxidation potentials than that used for the detection of hydrogen peroxide the interference from electroactive species (e.g., ascorbic and uric acids present) in clinical samples such as blood or serum is greatly reduced. For example ferrocene derivatives have oxidation potentials in the +0.1 to 0.4 V range. Conductive organic salts such as tetrathiafulvalene-tetracyanoquinodimethane (TTF-TCNQ) can operate as low as 0.0 Volts relative to a Ag/AgCl reference electrode. Nankai et al, WO 86/07632, published Dec. 31, 1986, discloses an amperometric biosensor system in which a fluid containing glucose is contacted with glucose oxidase and potassium ferricyanide. The glucose is oxidized and the ferricyanide is reduced to ferrocyanide. This reaction is catalyzed by glucose oxidase. After two minutes, an electrical potential is applied, and a current caused by the re-oxidation of the ferrocyanide to ferricyanide is obtained. The current value, obtained a few seconds after the potential is applied, correlates to the concentration of glucose in the fluid.

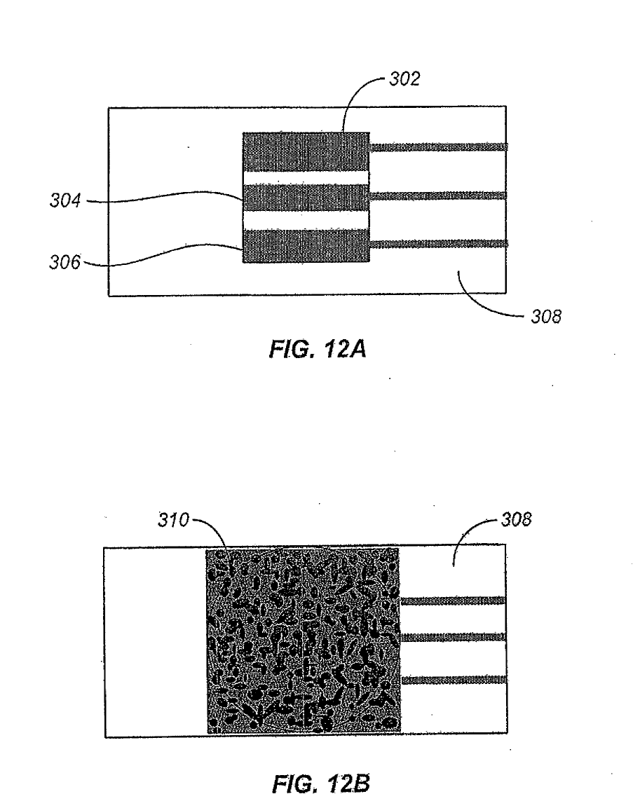

There are multiple glucose sensors that may be used with this invention. In a three electrode system, shown in FIG. 12 a working electrode 302, such as Pt, C, or Pt/C is referenced against a reference electrode 304 (such as Ag/AgCl) and a counter electrode 306, such as Pt, provides a means for current flow. The three electrodes are mounted on a substrate 308 then covered with a reagent 310, as shown in FIG. 12(b).

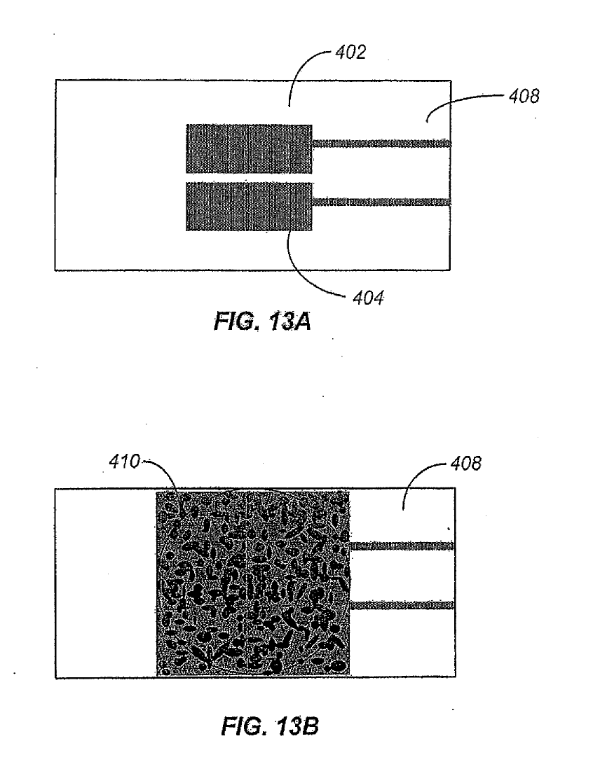

FIG. 13 shows a two electrode system, wherein the working and auxiliary electrodes 402 and 404 are made of different electrically conducting materials. Like the embodiment of FIG. 12, the electrodes 402 and 404 are mounted on a flexible substrate 408 as shown in FIG. 13 and covered with a reagent 410, as shown in FIG. 13(b). In an alternative two electrode system, the working and auxiliary electrodes are made of the same electrically conducting materials, where the reagent exposed surface area of the auxiliary electrode is slightly larger than that of the working electrode or where both the working and auxiliary electrodes are substantially of equal dimensions.

In amperometric and coulometric biosensors, immobilization of the enzymes is also very important. Conventional methods of enzyme immobilization include covalent binding, physical adsorption or cross-linking to a suitable matrix may be used.

In some embodiments, the reagent is contained in a reagent well in the biosensor. The reagent includes a redox mediator, an enzyme, and a buffer, and covers substantially equal surface areas of portions of the working and auxiliary electrodes. When a sample containing the analyte to be measured, in this case glucose, comes into contact with the glucose biosensor the analyte is oxidized, and simultaneously the mediator is reduced. After the reaction is complete, an electrical potential difference is applied between the electrodes. In general the amount of oxidized form of the redox mediator at the auxiliary electrode and the applied potential difference must be sufficient to cause diffusion limited electrooxidation of the reduced form of the redox mediator at the surface of the working electrode. After a short time delay, the current produced by the electrooxidation of the reduced form of the redox mediator is measured and correlated to the amount of the analyte concentration in the sample. In some cases, the analyte sought to be measured may be reduced and the redox mediator may be oxidized.

In the present invention, these requirements are satisfied by employing a readily reversible redox mediator and using a reagent with the oxidized form of the redox mediator in an amount sufficient to insure that the diffusion current produced is limited by the oxidation of the reduced form of the redox mediator at the working electrode surface. For current produced during electrooxidation to be limited by the oxidation of the reduced form of the redox mediator at the working electrode surface, the amount of the oxidized form of the redox mediator at the surface of the auxiliary electrode must always exceed the amount of the reduced form of the redox mediator at the surface of the working electrode. Importantly, when the reagent includes an excess of the oxidized form of the redox mediator, as described below, the working and auxiliary electrodes may be substantially the same size or unequal size as well as made of the same or different electrically conducting material or different conducting materials. From a cost perspective the ability to utilize electrodes that are fabricated from substantially the same material represents an important advantage for inexpensive biosensors.

As explained above, the redox mediator must be readily reversible, and the oxidized form of the redox mediator must be of sufficient type to receive at least one electron from the reaction involving enzyme, analyte, and oxidized form of the redox mediator. For example, when glucose is the analyte to be measured and glucose oxidase is the enzyme, ferricyanide or quinone may be the oxidized form of the redox mediator. Other examples of enzymes and redox mediators (oxidized form) that may be used in measuring particular analytes by the present invention are ferrocene and or ferrocene derivative, ferricyanide, and viologens. Buffers may be used to provide a preferred pH range from about 4 to 8. The most preferred pH range is from about 6 to 7. The most preferred buffer is phosphate (e.g., potassium phosphate) from about 0.01 M to 0.5 M and preferably about 0.05 M. (These concentration ranges refer to the reagent composition before it is dried onto the electrode surfaces.) More details regarding glucose sensor chemistry and operation may be found in: Clark L C, and Lyons C, “Electrode Systems for Continuous Monitoring in Cardiovascular Surgery,” Ann NY Acad Sci, 102:29, 1962; Updike S J, and Hicks G P, “The Enzyme Electrode,” Nature, 214:986, 1967; Cass, A. E. G., G. Davis. G. D. Francis, et. al. 1984. Ferrocene-mediated enzyme electrode for amperometric determination of glucose. Anal. Chem. 56:667-671; and Boutelle, M. G., C. Stanford. M. Fillenz, et al. 1986. An amperometric enzyme electrode for monitoring brain glucose in the freely moving rat. Neurosci lett. 72:283-288.

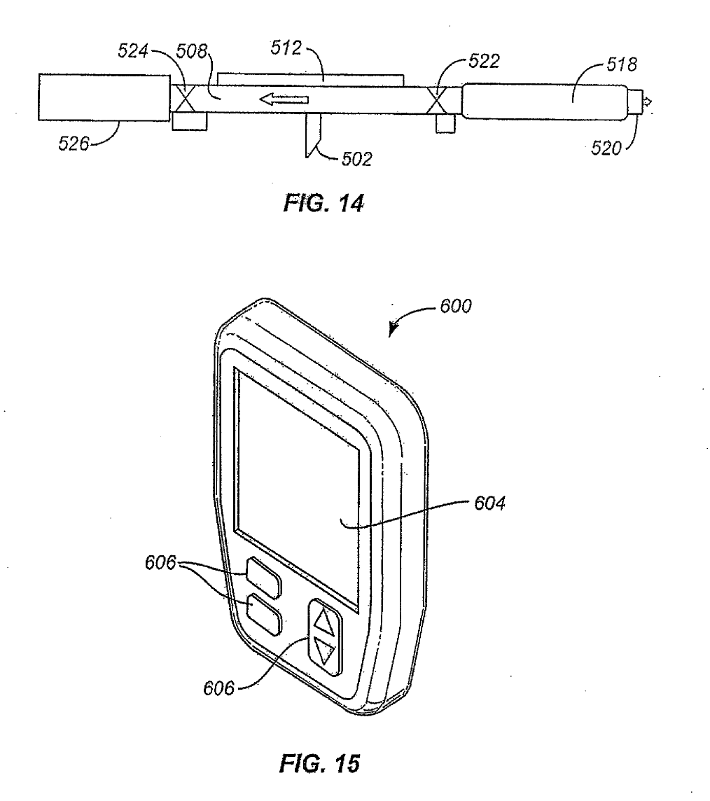

Another embodiment of the disposable portion of the glucose monitor invention is shown in FIG. 14 with a needle 502 and a glucose sensor 512 in fluid communication with a sensing area in channel 508. In this embodiment, actuator 520 is on the side of sensing fluid reservoir 518, and the waste reservoir 526 is expandable. Operation of actuator 520 sends sensing fluid from reservoir 518 through one way flap valve 522 into the sensing area in channel 508 and forces sensing fluid within channel 508 through flap valve 524 into the expandable waste reservoir 526. While one needle is shown more than one needle may be used. Alternatively, a deformed substrate layer as described herein or other types of microneedle arrays may be used in the glucose monitor of FIG. 14.

In some of the embodiments described herein, the starting amount of sensing fluid in a sensing fluid reservoir is about 1.0 ml or less, and operation of the sensing fluid actuator sends about 5 μL to about 25 μL of fresh sensing fluid into the sensing channel. Recalibrating the device three times a day for seven days will use less than about 1000 μL of sensing fluid.

FIGS. 15 and 16 show a remote receiver for use with a glucose monitoring system. The wireless receiver can be configured to be worn by a patient on a belt, or carried in a pocket or purse. In this embodiment, glucose sensor information is transmitted by the glucose sensor 602 applied to the user's skin to receiver 600 using, e.g., wireless communication such as radio frequency (RF) or Bluetooth wireless. The receiver may maintain a continuous link with the sensor, or it may periodically receive information from the sensor. The sensor and its receiver may be synchronized using RFID technology or other unique identifiers. Receiver 600 may be provided with a display 604 and user controls 606. The display may show, e.g., glucose values, directional glucose trend arrows and rates of change of glucose concentration. The receiver can also be configured with a speaker adapted to deliver an audible alarm, such as high and low glucose alarms. Additionally, the receiver can include a memory device, such as a chip, that is capable of storing glucose data for analysis by the user or by a health care provider.

The monitor, preferably the wireless receiver component, can be programmed with high and low threshold levels such that when the patient's glucose levels are higher than the high threshold level or lower than the low threshold level the monitor will alert the patient or a third party. The receiver can be preprogrammed to default threshold levels, can be manually programmed using, for example, the receiver's user interface, or the receiver can be adapted to dynamically adjust threshold levels based on, for example, current glucose concentrations, trends in the glucose concentrations, or user inputs into the receiver such as an indication from the user that she is going to sleep or about to consume food. The alert can occur based on any method to alert the patient, such as, for example, with an audible alert like a beep, a visual alert such as a blinking light, or mechanical alert such as vibrating. The monitor can also be adapted to wirelessly alert a device separate from the receiver, such as a health care provider, when the glucose concentration is above or below the threshold levels, or trending below or above the threshold levels. The monitor, and preferably the receiver, can also be adapted to display glucose concentration trends and can alert the patient′ when the concentration is trending down or up. Trends can be stored in the receiver and can be used to dynamically adjust the threshold levels.

In some embodiments, the source reservoir for the calibration and sensing fluid may be in a blister pack which maintains its integrity until punctured or broken. The actuator may be a small syringe or pump. Use of the actuator for recalibration of the sensor may be performed manually by the user or may be performed automatically by the device if programmed accordingly. There may also be a spring or other loading mechanism within the reusable housing that can be activated to push the disposable portion—and specifically the microneedles—downward into the user's skin.

Referring to FIGS. 17-26, further embodiments of the invention are depicted having microneedles with diverse configurations. In the example shown in FIG. 17, a slender pyramid-shaped microneedle 700 is formed on a substrate 710. Microneedle 700 includes a lumen having a distal opening 720 along one edge of the pyramid. The lumen extends from the distal opening 720 through the microneedle 700 to a proximal opening (not shown) on the opposite side of substrate 710. Substrate 710 with a single microneedle 700, or an array of microneedles 700, may be used with the analyte monitors described above to penetrate tissue for analyte sampling.

Referring to FIG. 18, a microneedle 730 is shown having a configuration different from that of microneedle 700 shown in FIG. 17. Microneedle 730 can be referred to as a “skinny sharp”, as its smaller cross-section forms a sharper point than that of microneedle 700. Microneedle 730 may or may not be provided with a lumen therethrough.

Many other variations of microneedles may be formed. Each configuration of microneedle has its own advantages and disadvantages. The slender pyramid 700 for instance lends itself well to having a through-lumen on one side or edge, as shown in FIG. 17. However, this configuration may not be ideal for tissue penetration. For tissue penetration, Applicants have found that a skinny sharp microneedle 730 is more ideal. Forming a lumen through the skinny sharp 730 while maintaining its toughness, however, is difficult.

According to aspects of the present invention, Applicants have found that the unique advantages of two or more microneedle configurations can be utilized by creating a mixed array of microneedles. For example, a slender pyramid 700 can be surrounded with a number of skinny sharps 730, as shown in FIG. 19, to help in tissue penetration. The skinny sharps 730 easily penetrate skin and stretch the skin enough to make penetration of the slender pyramid 700 easy.

Microneedles having different configurations may be formed on the same substrate 710, as shown in FIG. 19, or on different substrates. In some embodiments of the invention, configuration elements that may be different from one type of microneedle to the other include size (for example variations in height, width, depth, radius), shape (for example cross-sections that are circular, square, triangular, hexagonal, non-symmetrical), orientation and the material the microneedle. The presence or absence of a lumen may be different, as described above, or the quantity or characteristics of the lumens may be different. For example, it may be desirable in some circumstances to combine microneedles having two lumens and/or a slotted lumen with microneedles having a single, circular cross-section lumen. By combining at least two different types of microneedles in this fashion, a microneedle array can be provided that has overall properties that are superior to those of an array having a single needle type.

Dimensions of the microneedles in some embodiments are as follows:

| | | |
|---|---|---|
| Needle size at the base | 125-250 | μm |
| Needle Height | 250-450 | μm |
| Proximal Lumen Opening | 30-80 | μm |
| Distal Lumen Opening | 10-30 | μm |
| Pitch (needle center to center) | 250-400 | μm |

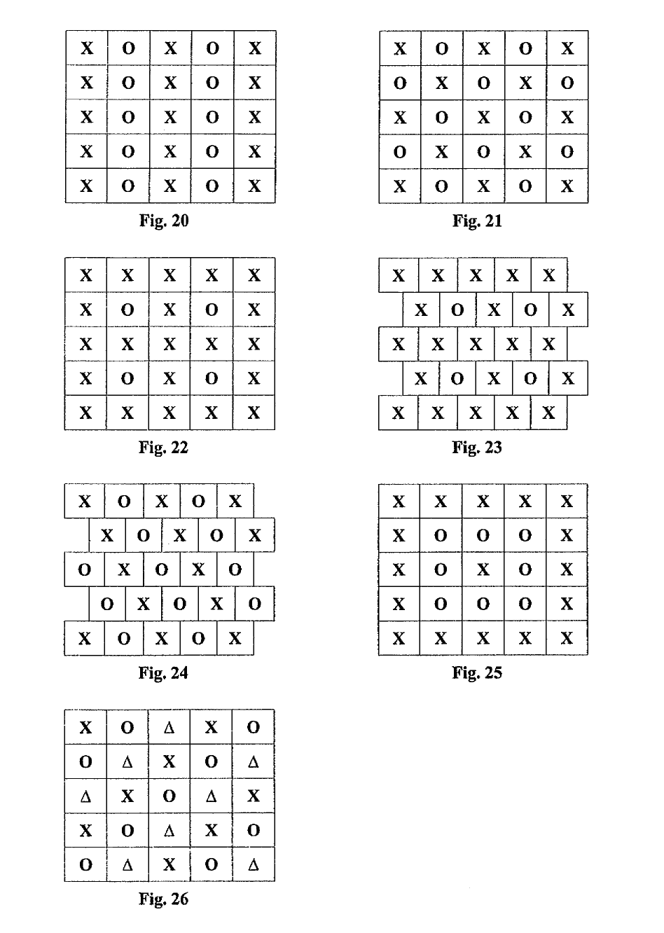

Referring to FIGS. 20-26, various examples of microneedle array patterns according to aspects of the present invention are depicted. In these figures, the “X”s are meant to depict a plurality of first tissue piercing elements (for instance skinny sharps 730, as shown in FIG. 18) having a common first configuration. Similarly, the “O”s are meant to depict a plurality of second tissue piercing elements (for instance slender pyramids 700, as shown in FIG. 17) having a common second configuration which is substantially different than the first configuration. In FIG. 26, the “Δ”s are meant to depict yet a third type of tissue piercing elements.

Referring now in particular to FIG. 20, an array of microneedles is depicted wherein columns of skinny sharps 730 (“X”s) are interleaved with columns of slender pyramids 700 (“O”s). FIG. 21 depicts a checkerboard array wherein the two types of microneedles are interspersed. FIG. 22 depicts a square array wherein each slender pyramid 700 (“O”) is surrounded by nine skinny sharps 730 (“X”s). FIG. 23 depicts a hexagonal array wherein each slender pyramid 700 (“O”) is surrounded by six skinny sharps 730 (“X”s). FIG. 24 depicts a diagonal array. FIG. 25 depicts a concentric array. FIG. 26 depicts a tri-configuration array having three different types of microneedles or tissue piercing elements. Other types of arrays (not depicted) that may be advantageous in certain situations include circular arrays random pattern arrays. While exemplary FIGS. 20-26 each depict an array having 25 tissue piercing elements, arrays have greater or fewer tissue piercing elements may be employed.

What is meant by “tissue piercing element” is an element that pierces, punctures, cuts or otherwise penetrates a tissue surface.

What is meant by a configuration that is “substantially different” than another configuration is one having characteristics that are distinguishable beyond mere differences due to manufacturing tolerances and process variations.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A surface penetration device comprising:
a substrate having a first surface and a second surface;
a plurality of first tissue piercing elements extending from and supported by the first surface of the substrate; the first tissue piercing elements having a common first configuration; and
a plurality of second tissue piercing elements extending from and supported by the first surface of the substrate; the second tissue piercing elements having a common second configuration, wherein the second configuration is substantially different than the first configuration, wherein at least some of the tissue piercing elements have a distal opening, a proximal opening, and a lumen extending between the distal and proximal openings, and wherein the proximal openings are in fluid communication with an opening in the second surface of the substrate.

2. A surface penetration device according to claim 1 further comprising a plurality of third tissue piercing elements extending from and supported by the first surface of the substrate; the third tissue piercing elements having a common third configuration, wherein the third configuration is substantially different than the first configuration and the second configuration.

3. A surface penetration device according to claim 1, wherein at least the first tissue piercing elements are micro-needles.

4. A surface penetration device according to claim 1, wherein at least the first tissue piercing elements are arranged in an array having a square format.

5. A surface penetration device according to claim 1, wherein at least the first tissue piercing elements are arranged in an array having a hexagonal format.

6. A surface penetration device according to claim 1, wherein the first tissue piercing elements comprise lumens and the second tissue piercing elements do not comprise lumens.

7. A surface penetration device according to claim 1, wherein both the first and the second tissue piercing elements comprise lumens.

8. A surface penetration device according to claim 1, wherein the first configuration of the first tissue piercing elements comprises a slender pyramid.

9. A surface penetration device according to claim 1, wherein the first configuration of the first tissue piercing elements comprises a skinny sharp needle.

10. A surface penetration device according to claim 1, wherein the first configuration of the first tissue piercing elements comprises a slender pyramid and the second configuration of the second tissue piercing elements comprises a skinny sharp needle.

11. A surface penetration device according to claim 10, wherein at least one of the first tissue piercing elements is surrounded by a plurality of second tissue piercing elements.

12. An analyte monitor comprising:
at least one first tissue piercing element having a first configuration;
at least one second tissue piercing element having a second configuration, wherein the second configuration is substantially different than the first configuration, wherein at least one of the tissue piercing elements has a distal opening, a proximal opening, and a lumen extending between the distal and proximal opening;
a sensing area in fluid communication with the proximal opening of the tissue piercing element; and
an analyte sensor adapted to detect an analyte in a fluid located within the sensing area.

13. An analyte monitor according to claim 12 further comprising at least one third tissue piercing element extending from and supported by the first surface of the substrate; the third tissue piercing element having a third configuration, wherein the third configuration is substantially different than the first configuration and the second configuration.

14. An analyte monitor according to claim 12, wherein at least the first tissue piercing element is a micro-needle.

15. An analyte monitor according to claim 12, comprising a plurality of first tissue piercing elements arranged in an array having a square format.

16. An analyte monitor according to claim 12, comprising a plurality of first tissue piercing elements arranged in an array having a hexagonal format.

17. An analyte monitor according to claim 12, wherein the first tissue piercing element comprises a lumen and the second tissue piercing element does not comprise a lumen.

18. An analyte monitor according to claim 12, wherein both the first and the second tissue piercing elements comprise lumens.

19. A method of penetrating tissue comprising:
providing a surface penetration device that comprises:
a substrate having a first surface and a second surface;
a plurality of first tissue piercing elements extending from and supported by the first surface of the substrate; the first tissue piercing elements having a common first configuration; and
a plurality of second tissue piercing elements extending from and supported by the first surface of the substrate; the second tissue piercing elements having a common second configuration, wherein the second configuration is substantially different than the first configuration, wherein at least some of the tissue piercing elements have a distal opening, a proximal opening, and a lumen extending between the distal and proximal openings, and wherein the proximal openings are in fluid communication with an opening in the second surface of the substrate; and
urging the surface penetration device against a tissue surface until at least some of the first tissue piercing elements and at least some of the second tissue piercing elements penetrate the tissue surface.

20. A tissue penetrating method according to claim 19, wherein the surface penetration device is urged against the tissue surface until at least some of the distal openings of the tissue piercing elements come in contact with interstitial fluid.
